(19) United States
(12) Patent Application Publication
(10) Pub. No.: US 2010/0241197 A1
(43) Pub. Date: Sep. 23, 2010

(54) METHODS OF USING NITROXIDES IN CONJUNCTION WITH PHOTOSENSITIZERS AND SONOSENSITIZERS

(75) Inventors: Kenneth Chang, Cerritos, CA (US); Louis Habash, Newport Coast, CA (US)

Correspondence Address: KNOBBE MARTENS OLSON & BEAR LLP, 2040 MAIN STREET, FOURTEENTH FLOOR, IRVINE, CA, 92614, US

(73) Assignee: Mitos Pharmaceuticals, Inc., Newport Beach, CA (US)

(21) Appl. No.: 12/794,584

(22) Filed: Jun. 4, 2010

Related U.S. Application Data
(63) Continuation of application No. 10/558,599, filed on Nov. 29, 2005.
(60) Provisional application No. 60/474,387, filed on May 29, 2003.

(51) Int. Cl. A61N 5/06; A61K 31/409; A61K 31/445; A61N 7/00

(52) U.S. Cl. 607/88; 514/410; 514/315; 601/2

(57) ABSTRACT

The teachings herein relate to new methods of ameliorating the negative effects of a photosensitizer or sonosensitizer in a patient using a nitroxide.

### CROSS-REFERENCE TO RELATED APPLICATIONS

The present application is a continuation of U.S. patent application Ser. No. 10/558,599 filed Nov. 29, 2005, which is a U.S. National Phase of International Application No. PCT/US2004/16190, filed May 21, 2004, which claims the benefit of U.S. Provisional Application No. 60/474,387 filed May 29, 2003 which are expressly incorporated by reference in their entireties.

### FIELD OF THE INVENTION

This invention relates to the use of one or more nitroxides in conjunction with photosensitizers and sonosensitizers. In particular the embodiments herein relate to methods of using one or more nitroxides to counteract the negative effects of photosensitizers or sonosensitizers being used as therapeutics and diagnostics.

### BACKGROUND OF THE INVENTION

Phototherapy is a term which includes all treatments that use light to induce reactions in the body which are of benefit to patients. Photodynamic therapy (PDT), a specific form of phototherapy, is a developing technique which can destroy unwanted tissue (e.g., tumor), while sparing normal healthy tissue. In general, PDT utilizes a photosensitizing compound (photosensitizer) which is a molecule having the ability to absorb light energy and use this energy to carry out chemical reactions in cells and body tissues. A photosensitizer alone is generally harmless and has no effect on either healthy or abnormal tissue. However, when a particular wavelength of light is directed onto tissue containing the photosensitizer, the photosensitizer becomes activated and, in the case of PDT, the tissue is rapidly destroyed, but generally only precisely where the light has been directed. Thus, by careful application of the light beam, this technique can be used as a localized treatment to selectively target abnormal tissue, such as a tumor, for example.

Modern photodynamic therapy most likely began with Schwartz and Lippman's work in the 1960s. Schwartz obtained a preparation derived from haematoporphyrin (Hp) which was shown, by fluorescence measurements, to concentrate at neoplastic loci. Since the structure of the product was unknown, it was termed “haematoporphyrin derivative” (HpD). This resulted in the supposition that HpD was a single compound, and has resulted in some confusion in the literature. It is now known that HpD contains a large number of products based on the porphyrin structure. There is a mixture of monomers consisting of Hp, protoporphyrin (Pp) and an intermediate dehydration product, hydroxyethyl vinyl deuteroporphyrin (HVD). The final step in HpD preparation involves solubilization of the product in dilute base. This results in a variety of unexpected reactions, including formation of dimers and higher oligomers. A current, commercially-available clinical photosensitizer is PHOTOFRIN® (available from Axcan Pharma Inc., Quebec Canada) a preparation with the concentration of monomers and unstable components greatly reduced.

The broad-brush clinical experiments of Dougherty et al. (1978) were followed by reports from various clinical laboratories on the utilization of HpD and its commercial extensions, particularly PHOTOFRIN®, in the treatment of solid tumors. Treatments of cancer of the aerodigestive tract, superficial bladder cancer, gynecological cancer and skin cancer were reported, generally with positive results. For example, in Japan, Hayata and Kato and their colleagues reported clinical results on the PDT of lung cancer in 1982. These workers used HpD, and later PHOTOFRIN®, in their studies. In the initial investigation of 299 lesions at various stages of development, 134 were judged to show complete remission. This work was followed up by detailed studies on early-stage central type squamous cell carcinoma, intraoperative studies, palliative studies, and a multicenter phase II clinical trial for early-stage lung cancer. The results were encouraging and the procedure was cost-effective.

Even by the early 1980s there was already work in progress to produce improved photosensitizers, improved, that is with respect to HpD and its relatives. The HpD drugs became known as “first generation”; the new ones were termed “second generation”. Some of the photosensitizer drugs being developed also have the desirable property of concentrating in tumors (and certain other kinds of proliferating tissue) relative to the surrounding healthy tissue, which also helps in targeting. It is now clear that there are some indications where PDT is at least as good as and possibly better than alternative treatments. Currently PDT is being used to treat cancers, and diseases, such as age-related macular degeneration (AMD), which is often caused by choroidal neovascularization.

The following provides a background on the general mechanics behind PDT and photodiagnosis. Typically, the photosensitizer is administered (e.g., injected, orally, topically) to a subject and allowed to circulate in the subject's system. After the photosensitizer is activated by a light source, the photosensitizing agent is converted to the triplet stage, shifting an electron to a higher orbital and thus generating two unpaired electrons. After the photosensitizer becomes excited it can then collide with biological molecules. There are generally two mechanisms by which the triplet state photosensitizer can react with biomolecules: Type I and Type II reactions. Type I reactions are radical mediated and Type II produce electronically excited and highly reactive singlet oxygen.

More specifically, a Type I reaction generally involves electron/hydrogen transfer directly from the photosensitizer, producing ions, or electron/hydrogen abstraction from a substrate molecule to form free radicals. These radicals then react rapidly, usually with oxygen, resulting in the production of highly reactive oxygen species (e.g. the superoxide and the peroxide anions). These radicals then attack cellular targets. Free radical attack on a cellular membrane (e.g., tumor cell) results in a loss of membrane integrity, followed by necrotic death. Cytosolic cytochrome C and high intracellular Ca2+ can then activate the caspase cascade, resulting in DNA cleavage and the destruction of intracellular proteins.

In a Type II reaction, the transfer of energy from the activated photosensitizer to molecular oxygen results in the formation of highly toxic reactive oxygen species, a singlet oxygen. Typically these oxygen species can kill targeted cells either by necrotic mechanisms or by initiating the apoptotic cascade.

In PDT, it is difficult to distinguish between the two reaction mechanisms. Even in cases where Type I reactions may occur, the Type II reaction will usually take place in tandem and it is difficult to differentiate the photobiological effects which are exclusively due to radical species. While PDT works by generating free radicals to target the cancerous cells, these free radicals can cause extensive damage to healthy normal tissue, cells, and subcellular organelles and molecules if the photosensitizers are activated away from the target site.

In addition to being used in PDT, photosensitizers have also been used for diagnostic purposes, such as fluorescent markers for example Like PDT, photodiagnosis can also utilize photosensitizers that absorb a particular wavelength of light. In the case of fluorescence and phosphorescence photodiagnosis, the absorbed photon from the illuminating radiation excites a photosensitizer's electron from a ground state to a higher state. The excited electron then falls to a lower level, but not immediately back to the ground state, emitting a longer-wavelength photon than it absorbed thereby allowing a practitioner to easily differentiate the cancer cells from the normal tissue by using a fluorescence scanner, for example.

Unfortunately, many photosensitizers have the main disadvantage of having long-term skin phototoxicity. Other drawbacks from using photosensitizers as a therapeutic or diagnostic can include very long (e.g., 2.5-3 months) clearance periods from the body, mainly due to large systemically introduced doses and high non-specific affinity to normal proteins and glycoproteins, a low PDT efficacy connected with low yields of singlet oxygen as a consequence of little 630 nm light penetration to tissues, and a considerable affinity to epithelial tissues, resulting in the red color of skin during and after treatment, accompanied by increased skin sensitivity to the daylight. Furthermore, practitioners generally wait 24-72 hours between introduction of the photosensitizers to a patient and irradiation of the tumor lesion. During this interim, patients typically stay in a darkened room.

Because of these negative side effects, patients typically avoid bright light for some time following the administration of the drug, oftentimes weeks after irradiation is applied. This can be a great inconvenience for people who work outside or who are often exposed to sunlight for long periods of time. While sunscreen and appropriate clothing (e.g. long-sleeve shirts) can help prevent activation of the photosensitizers, there is a need in the art to help prevent the negative results to the body and skin if the photosensitizers become activated away from the target site.

### SUMMARY OF THE INVENTION

Certain embodiments herein are directed to methods of treating a patient, including administering a sufficient amount of a nitroxide to prevent or treat a negative side effect resulting from photosensitizers or sonosensitizers. In further embodiments, the methods herein can be applied to photosensitizers used in photodynamic therapy. In more specific embodiments, the photosensitizer can be selected from: porphyrins, chlorins, phthalocyanines, and the like, for example. In even more particular embodiments, the photosensitizer can be PHOTOFRIN®. In further embodiments, the methods herein can ameliorate negative effects such as oxidative stress, photosensitivity, generalized toxicity, and cellular apoptosis. In further embodiments, the nitroxide to be used with the methods herein can be 4-hydroxy-2,2,6,6-tetramethylpiperidine-1-oxyl.

Further embodiments, include methods of treating a patient suffering from cancer, including systematically administering a therapeutically effective amount of photosensitizer to said patient; applying light to a target region on the patient; wherein the light has a wavelength that sufficiently penetrates the patient and activates the administered photosensitizer; and administering a therapeutically effective amount of a nitroxide to the patient, such that a negative effect of the photosensitizer is ameliorated. In more specific embodiments, the photosensitizer can be selected from the group consisting of porphyrins, chlorins, and phthalocyanines, and the like. In even more particular embodiments, the photosensitizer can be PHOTOFRIN®. In particular embodiments, the methods herein can be used in the treatment of lung, breast, and skin cancer. In further embodiments, the methods herein can ameliorate negative effects such as oxidative stress, photosensitivity, generalized toxicity, and cellular apoptosis.

Other embodiments, include methods of diagnosing a patient suspected of having cancer, including systematically administering a sufficient amount of a photosensitizer to the patient; wherein the photosensitizer has a high specificity for cancerous cells and is capable of emitting a detectable wavelength of light when activated by a particular wavelength; activating the administered photosensitizer with the particular wavelength of light; detecting the photosensitizer's emitted wavelength; administering a therapeutically effective amount of a nitroxide to the patient, such that a negative effect of the photosensitizer is ameliorated. In particular embodiments, the methods herein can be used in the diagnosis of lung, breast, and skin cancer. In further embodiments, the methods herein can ameliorate negative effects such as oxidative stress, photosensitivity, generalized toxicity, and cellular apoptosis. In further embodiments, the nitroxide to be used with the methods herein can be 4-hydroxy-2,2,6,6-tetramethylpiperidine-1-oxyl.

Further embodiments, include methods of treating a patient suffering from cancer, including systematically administering a therapeutically effective amount of sonosensitizer to said patient; applying ultrasound or sonoluminescence to a target region on the patient; wherein the ultrasound or sonoluminescence has a wavelength that sufficiently penetrates the patient and activates the administered sonosensitizer; administering a therapeutically effective amount of a nitroxide to the patient, such that a negative effect of the sonosensitizer is ameliorated. In particular embodiments, the methods herein can be used in the treatment of lung, breast, and skin cancer. In further embodiments, the methods herein can ameliorate negative effects such as oxidative stress, photosensitivity, generalized toxicity, and cellular apoptosis. In further embodiments, the nitroxide to be used with the methods herein can be 4-hydroxy-2,2,6,6-tetramethylpiperidine-1-oxyl.

Additional embodiments include the use of a nitroxide in the preparation of a medicament to prevent or treat a negative side effect resulting from a photosensitizer or sonosensitizer through administration to a mammalian patient. In further embodiments, the uses herein can be applied to photosensitizers used in photodynamic therapy. In more specific embodiments, the photosensitizer can be selected from: porphyrins, chlorins, phthalocyanines, and the like, for example. In even more particular embodiments, the photosensitizer can be PHOTOFRIN®. In further embodiments, the uses herein can ameliorate negative effects such as oxidative stress, photosensitivity, generalized toxicity, and cellular apoptosis. In further embodiments, the nitroxide to be used with the teachings herein can be 4-hydroxy-2,2,6,6-tetramethylpiperidine-1-oxyl.

Other embodiments include medicaments for preventing or treating a negative side effect resulting from a photosensitizer or sonosensitizer, wherein said medicament comprises a nitroxide. In further embodiments, the medicaments herein can be applied to patients who have been administered photosensitizers during photodynamic therapy. In more specific embodiments, the photosensitizer can be selected from: porphyrins, chlorins, phthalocyanines, and the like, for example. In even more particular embodiments, the photosensitizer can be PHOTOFRIN®. In further embodiments, the medicaments herein can ameliorate negative effects such as oxidative stress, photosensitivity, generalized toxicity, and cellular apoptosis. In further embodiments, the nitroxide to be used with the medicaments herein can be 4-hydroxy-2,2,6,6-tetramethylpiperidine-1-oxyl.

### DETAILED DESCRIPTION OF THE PREFERRED EMBODIMENT

### Photosensitizers

The methods disclosed herein include the use of a nitroxide to ameliorate the negative effects of any photosensitizer in a patient's body. The term “photosensitizer” as used herein, is to be construed broadly and generally relates to an agent that is capable of absorbing light energy and using that energy to carry out chemical reactions in cells and body tissues. In certain embodiments, the term “photosensitizer” relates to an agent that generates free radicals, including reactive oxygen species. In further embodiments the term “photosensitizer” relates to a marker used in photodiagnosis, such as a fluorescent marker for example. Many patents including U.S. Pat. No. 4,977,177 to Bommer et al., and U.S. Pat. No. 5,591,847, U.S. Pat. No. 5,770,730, and Australian Patent, 669,876, issued to Pandey et. al, disclose photosensitizers, and are hereby expressly incorporated by reference in their entireties. Any photosensitizer, including those described in these patents, can be used with the methods herein.

There are three main classes of photosensitizing compounds: porphyrins, chlorins, and phthalocyanines. Other types of photosensitizers include modified porphyrins, bacteriochlorins, naphthalocyanines, pheophorbides and purpurins, for example. The methods described herein are capable of ameliorating negative effects caused by any photosensitizers, including each photosensitizer described herein, for example. Because PDT and photodiagnostics are developing fields, it is important to note that the methods disclosed herein are capable of ameliorating, to at least some extent, the negative effects caused by any currently available photosensitizer and any photosensitizer that becomes available in the future.

In some embodiments, the methods disclosed herein are capable of ameliorating the negative effects caused by porphyrins, including Hpd and its derivatives PHOTOFRIN® (available from Axcan Pharma Inc., Quebec Canada), and PHOTOFRIN II, for example. PHOTOFRIN® has shown to be effective in treating cancers of the lung, esophagus, stomach, cervix, and pre-cancerous conditions of the cervix, and the like. PHOTOFRIN® is generally activated by red light at around 630 nm.

In further embodiments, the methods disclosed herein are capable of ameliorating the negative effects caused by chlorins or bacteriochlorins. In general, chlorins are characterized by having one of the exo-pyrrole double bonds of the porphyrin ring hydrogenated, resulting in an intense absorption at wavelengths greater than 650 nm. In bacteriochlorins, two of the exo-pyrrole double bonds of the porphyrin ring are hydrogenated, yielding compounds with maximum absorption at even longer wavelengths. Because of these improved optical properties, chlorins and bacteriochlorins are being intensively developed as new drugs for PDT. Furthermore, because cholins are activated at higher wavelengths (e.g. wavelengths that can more easily penetrate a patient's skin), they allow for treatment of deeper tumors than polyphrins such as HpD and the like. Examples of chlorins and bacteriochlorins that can be ameliorated with the methods disclosed herein include bonellin, Meta-tetra hydroxyphenyl chlorin (m-THPC), Mono-L-aspartyl chlorin e6 (NPe6 or MACE), and the like, for example.

In some embodiments, the methods herein can ameliorate the negative side effects caused by Meta-tetra hydroxyphenyl chlorin (m-THPC). m-THPC is a second generation photosensitizer, developed for clinical use by Scotia QuantaNova Ltd. m-THPC is also known as FOSCAN® (Biolitec AG, Germany). It has a hydrophobic chlorin core and hydroxyphenyl groups at the meso position to increase solubility of the photosensitizer. The first clinical study with m-THPC began in 1990 for the treatment of human mesothelioma and it is currently being developed for gynecological, respiratory and head and neck cancers in USA, Europe and the UK.

The advantages of m-THPC are highlighted by comparison with PHOTOFRIN®. m-THPC has been shown to be approximately 200 times more effective then PHOTOFRIN® when considering photodynamic dose (i.e. a lower photosensitizer dose and shorter illumination times are required to achieve similar results). m-THPC is a single pure compound, rather than a mixture of porphyrins. It is excited at a longer wavelength and the molar absorbance coefficient for m-THPC is much higher than that of PHOTOFRIN®, i.e. 22 400 M-1 cm-1 at 652 nm and 1 170 M-1 cm-1 at 630 nm respectively (in methanol). Furthermore, m-THPC has a longer half life in the triplet state generating more cytotoxic oxygen species, and is said to be more selective between tumor and normal tissue. Furthermore, m-THPC is more hydrophobic than PHOTOFRIN® which thus increases cellular uptake leading to higher efficacy in vitro. Despite these benefits over PHOTOFRIN®, the skin photosensitivity caused by m-THPC is only slightly less than that of PHOTOFRIN®.

In other embodiments, the methods described herein can be used to treat and prevent the negative effects caused by Mono-L-aspartyl chlorin e6 (NPe6 or MACE). Npe6 is a highly water soluble chlorin-type photosensitizer, and has an absorbance peak at 654 nm (extinction coefficient of 40 000 M-1 cm-1). Npe6 is generally effective in vitro and in vivo, shown by tumor retention and efficient photodynamic damage.

Still, in other embodiments, the present methods can be used to treat and prevent the negative effects of bacteriochlorins. Currently, a number of centers and companies are developing bacteriochlorins, which have very good optical properties in terms of tissue penetration. These compounds, which absorb light strongly above 740 nm, show remarkable potential as PDT agents.

In certain embodiments, the methods described herein can be used to prevent and treat the negative side effects caused by benzoporphyrin derivative mono-acid A (BPD). BPD is a chlorin-type molecule that has been developed by QuadraLogic Technologies. (Canada) It is a hydrophobic molecule that is distinguished by the presence of a mono-acid at either position 3 or 4 of the porphyrin ring. The absorbance peak for PDT typically occurs at 650 nm with an extinction coefficient of 34 000 M-1 cm-1. It has shown rapid tumor accumulation in clinical trials.

In yet further embodiments, the present methods can be used to treat and prevent the negative effects of phthalocyanines. Phthalocyanines are highly colored compounds which have found widespread commercial application and have been recently developed as photosensitizing agents for PDT.

In general, the pyrrole groups in phthalocyanines are conjugated to benzene rings and bridged by aza nitrogens rather than methine carbons. This causes the absorption spectrum to shift to longer wavelengths and the Q bands to become more intense than the Soret peak. The shift of this red absorption peak permits the use of longer wavelength light with increased tissue penetration to excite these compounds (typically around 680 nm), compared with the 630 nm light used to excite porphyrins.

The methods disclosed herein can also treat and prevent the negative effects of numerous other synthetic photosensitizers which have been developed with improved photophysical properties or tumor selectivity. These include: purpurins, porphycenes, pheophorbides, verdins, and the like, for example. Purpurins are a type of porphyrin macrocycle with an absorption band generally between 630 nm to 715 nm, typified by tin etiopurpurin (SnET2) which has an extinction coefficient of 40 000 M-1 cm-1 at 700 nm. Porphycenes, despite having activation wavelengths lower than other new photosensitizers (e.g., 635 nm), show fluorescence yields higher than HpD and are therefore likely photosensitizer candidates. Phorbides are derived from chlorophylls (e.g. pheophorbide) and have 20 times the effectiveness of HpD. Verdins contain a cyclohexanone ring fused to one of the pyrroles of the porphyrin ring and produce similar responses to HpD and purpurins.

In other embodiments, the methods described herein can ameliorate the negative effects caused by psoralens and their derivatives, which have been used for over 3000 years in the treatment of skin disorders and are still in use today. The cytotoxic action of these compounds stems from their ability to cross-link biomolecules, in particular DNA, following activation by ultraviolet light. In specific embodiments, the methods described herein can be used to ameliorate the negative effects of PUVA (psoralen with UVA) which is often used to treat psoriasis and other skin conditions.

In still further embodiments, the methods described herein can ameliorate the negative effects caused by anthracycline compounds which exhibit tumor selectivity. Members of this group include doxorubicin which is currently used in chemotherapy, although adverse side effects are common.

In other embodiments, the methods disclosed herein can ameliorate the negative effects caused by Lu-texaphyrin, SnEt2, tetrahydroxyphenylchlorin (THPC), and the like, for example.

In other embodiments, the methods described herein can treat and prevent synthetic non-porphyrin compounds that demonstrate photosensitizing ability. These compounds include: phenothiazinium compounds such as methylene blue; Toluidine blue, which has found widespread use in the diagnosis of oral disease; cyanines such as Merocyanine 540; acridine dyes as demonstrated by Raab in 1900; derivatives of the tumor marker, Nile blue; and rhodamines such as the mitochondria-specific Rhodamine 123.

In addition to treating and preventing the negative side effects of exogenous or synthetic photosensitizers, the methods described herein are capable of ameliorating the negative effects of endogenous photosensitizers, including porphyrins, such as protoporphyrin IX (PpIX), Heme, and the like, for example. 5-Aminolaevulinic acid (ALA) is a metabolic precursor in the biosynthesis of haem. The immediate precursor to haem in this pathway is PpIX which is a natural photosensitizer associated with some types of porphyria. The rate of formation of PpIX is dependent on the rate of synthesis of ALA from glycine and succinyl CoA which is governed in a negative-feedback manner by the concentration of free haem. Since the conversion of PpIX to haem is relatively slow, administration of exogenous ALA can bypass the negative-feedback mechanism and cause the build-up of phototoxic levels of the endogenous photosensitizer PpIX.

Because nitroxides can ameliorate the negative effects of endogenous porphyrins, the methods provided herein can be used to treat or prevent porphyria. Porphyria generally relates to a collection of related diseases in which porphyrins accumulate in various parts of the subject's body including the skin, bones and teeth, for example. These porphyrins can be transformed by sunlight into caustic toxins, and are capable of eating away tissue. In certain embodiments, the methods herein can be used to prevent and treat the following, non-exclusive list of effects caused by porphyria: tissue erosion, scars, dense pigmentation, anemia, and the like, for example.

The following is a non-exclusive list of photosensitizer brands that the methods described herein can be used with: PHOTOFRIN® (QLT, Vancouver, Canada), PHOTOFRIN II (QLT, Vancouver, Canada), PHOTOFLORA, PHOTOSENSE (Russia), PHOTOHEM Russia), VERTEPORFIN® (QLT, Vancouver, Canada), LUTRIN (Pharmacyclics, USA), FOSCAN (Biolitec AG, Germany), LEVULAN (Dusa Pharmaceuticals, Toronto, Canada), VISUDYNE (QLT and Novartis Opthalmics, Vancouver, Canada, and Duluth, Ga.), METVIX (Photocure, Oslo, Norway), PHOTOPOINT SnET2 (Miravant Medical Technologies, Santa Barbara, Calif.), PHOTOPOINT MV9411 (Miravant Medical Technologies, Santa Barbara, Calif.), ANTRIN (Pharmacyclics, Sunnyvale, Calif.), LUTRIN (Pharmacyclics, Sunnyvale, Calif.).

### Sonosensitizers

In addition to treating or preventing the negative effects caused by a photosensitzer, the present teachings also include methods of ameliorating the negative effects caused by any sonosensitizer. Sonosensitizers are similar to photosensitizers in that they are generally harmless in their pre-activated state, and can be used to treat unwanted cells with reactive oxidative species after they are activated. The main difference between the two types of compounds is that photosensitizers are typically activated by light energy and sonosensitizers are typically activated by ultrasound or sonoluminescence. Sonodynamic therapy (SDT) generally relates to the treatment of unwanted tissue with ultrasound or sonoluminescence, and typically involves sonosensitizers, an electrical power of 5 W/cm², and an irradiation of several minutes.

SDT and sonosensitizers are described in more detail in U.S. Pat. No. 6,498,945, issued to Alfheim, which is hereby expressly incorporated by reference in its entirety. The methods of prevention and treatment herein, can all be used with the methods and sonosensitizers disclosed in this patent. In certain embodiments, the sonosensitizers can be selected from water-soluble polymers (e.g., hexamers, higher polymers, and polyalkylene oxide compounds) and derivatives thereof, surfactants, oil-in-water emulsions, stabilized particles, certain chromophoric groups such as sulfonated dyes, and the like, for example. Because SDT is a developing field, it is important to note that the methods disclosed herein are capable of ameliorating the negative effects caused by any currently available sonosensitizer and any sonosensitizer that becomes available in the future.

### Nitroxides

The methods described herein are directed to the use of a nitroxide to ameliorate a negative side effect caused by a photosensitizer or sonosensitizer in a patient's body. In certain embodiments, the photosensitizer can be present in the patient's body as a result of phototherapy (e.g., PDT). In other embodiments, the photosensitizer is present as a result of photodiagnostics, for example. In other embodiments, the sonosensitizer is present in a patient's body as a result of SDT, for example.

As used herein the term “nitroxide” is to be construed broadly, and generally refers to stable free radical compounds that are capable of reacting with a variety of biologically relevant compounds such as free radicals, including oxy radicals, for example. In more specific embodiments, the nitroxides described herein are free radical scavengers or anti-oxidants.

Generally nitroxides can ameliorate most of the negative side effect that result from using photosensitizers and sonosensitizers. These effects include, but are not limited to, oxidative stress, skin phototoxicity, skin sensitivity, and damage caused to healthy cells by the formation of free radicals, including necrosis and apoptosis. Nitroxides can also prevent subcellular damage including damage to organelles and molecules such as DNA and RNA, and the like, for example.

According to certain embodiments, the nitroxides used in the methods described herein can be selected from the following formulas:

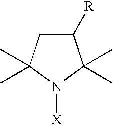

Wherein X is selected from O. and OH, and R is selected from COOH, CONH, CN, and CH₂NH₂

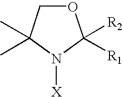

Wherein X is selected from O. and OH, and R₁ is selected from CH₃ and spirocylohexyl, and R₂ is selected from C₂H₅ and spirocyclohexyl

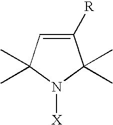

Wherein X is selected from O. and OH and R is selected from CONH.

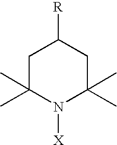

Wherein X is selected from O. and OH and R is selected from H, OH, and NH₂ and T is selected from O.

Other suitable nitroxides can be found in Proctor, U.S. Pat. No. 5,352,442, and Mitchell et al., U.S. Pat. No. 5,462,946, both of which are hereby incorporated by reference in their entireties.

A non-exclusive list of nitroxides that can be used with the methods described herein also include, 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO.

One preferred nitroxide that can be used with the methods described herein is Tempol, characterized by the chemical formula 4-hydroxy-2,2,6,6-tetramethylpiperidine-1-oxyl. Tempol is a stable nitroxide radical that can act as a free radical scavenger and therefore ameliorate the harmful effects that photosensitizers and sonosensitizers have on healthy cells or tissue in a subject.

In certain embodiments, a nitroxide can be used as a sole active ingredient in ameliorating the negative effects caused by photosensitizers and sonosensitizers. In other embodiments, a nitroxide can be used with other anti-oxidants, including other nitroxides, capable of stabilizing the harmful free radicals generated by photosensitizers and sonosensitizers in a subject's body as a result of phototherapy, photodiagnostics, and sonodynamic therapy. Other suitable anti-oxidants that can be used in conjunction with a nitroxide include, but are not limited to: Vitamins A, B, C, and E, selenium, isoflavones, polyphenols, carotenoids, carnosines, citric acid, phenolic compounds, BHA (butylated hydroxyanisole), BHT (butylated hydroxytoluene), propyl gallate, TBHQ (tert-butyl hydroquinone), lecithins, gum or resin guiac, THBP (trihydroxybutyrophenone), thiodipropionic acid, dilauryl thiodipropionate, co-enzyme Q10, alpha-lipoic acid, anthocyanins, beta carotene, catechins, ginkgo bilboa, lutien, lycopene, glutathione, proanthocyanidins, and the like, for example.

### Characteristics of Nitroxide Formulations

Nitroxides to be used herein, can be incorporated into any suitable formulation, or used alone. The particular nitroxide formulation to be used herein will depend on the intended method of administration, whether the mode of administration is oral, parenteral, including injection, or topical, and the like, for example. In certain embodiments, a nitroxide can be administered in the form of a pharmaceutical composition in combination with pharmaceutically acceptable carriers or excipients, the proportion and nature of which can be determined by the solubility and chemical properties of the nitroxide selected, the chosen route of administration, and standard pharmaceutical practice. In other embodiments, the nitroxides described herein, while effective themselves, can be formulated and administered in the form of their pharmaceutically acceptable salts, such as for example, acid addition salts, for purposes of stability, convenience of crystallization, increased solubility and the like.

A nitroxide utilized in accordance with the teachings herein can be administered in any form or mode which makes the nitroxide bioavailable, including oral, parenteral, and topical routes, and the like, for example. A non-exclusive list of administration routes include, oral, subcutaneous, intramuscular, intravenous, transdermal, intranasal, rectal, topical, and the like, for example. One skilled in the art of preparing formulations can readily select the proper form and mode of administration depending upon the particular characteristics of the nitroxide selected, after assessing the relevant circumstances.

In certain embodiments, a nitroxide can include a carrier or one or more excipients. In more specific embodiments, the carrier or excipient can be a solid, semi-solid, or liquid material which can serve as a vehicle or medium for the nitroxide. Suitable carriers or excipients are well known in the art. In further embodiments, a nitroxide can be adapted for oral, parenteral, or topical use and can be administered to the patient in the form of tablets, capsules, suppositories, solution, suspensions, or the like.

In certain embodiments, a nitroxide can be administered orally, for example, with an inert diluent or with an edible carrier. In other embodiments, a nitroxide can be enclosed in a gelatin capsule or compressed into a tablet. For certain embodiments directed to oral administration, a nitroxide can be incorporated with excipients and used in the form of tablets, troches, capsules, elixirs, suspensions, syrups, wafers, chewing gums and the like.

In other embodiments, nitroxide-containing tablets, pills, capsules, troches and the like can also include adjuvants typically utilized in the preparation of pharmaceuticals. For example, they can include one or more of the following adjuvants: binders such as microcrystalline cellulose, gum tragacanth or gelatin; excipients such as starch or lactose, disintegrating agents such as alginic acid, corn starch and the like; lubricants such as magnesium stearate or zinc stearate; glidants such as colloidal silicon dioxide; and sweetening agents such as sucrose or saccharin can be added or a flavoring agent, such as peppermint, methyl salicylate or orange flavoring, for example. When the dosage unit form is a capsule, it can contain, in addition to materials described above, a liquid carrier such as polyethylene glycol or a fatty oil, and the like, for example.

In other embodiments, the dosage unit forms can contain other materials which modify the physical form of the dosage unit, for example, as coatings. Thus, tablets or pills can be coated with sugar, shellac, or other enteric coating agents. In other embodiments, nitroxide-containing syrup can include a sweetening agent, such as sucrose, and certain preservatives, dyes and colorings and flavors, and the like, for example.

In certain embodiments, the nitroxides to be used with the methods described herein, are solutes dissolved in a suitable solvent. In other embodiments, the nitroxides to be used with the methods described herein can be in the form of a dispersion, suspension, liquid, thickened liquid, gel, or emulsion, for example. In additional embodiments, the nitroxide formulations are in the form of a cream, lotion, ointment and the like. Detail on how to prepare the above formulations is provided in Remington's Pharmaceutical Sciences, 18^{th} ed. 1990, which is hereby incorporated by reference in its entirety.

In further embodiments, nitroxide solutions or suspensions used for parenteral, intradermal, or subcutaneous application may include a sterile diluent such as water for injection, a saline solution, a fixed oil, a polyethylene glycol, glycerine, propylene glycol, other synthetic solvents, an antibacterial agent, such as benzyl alcohol or methyl paraben, an antioxidant such as ascorbic acid or sodium bisulfite, a chelating agent such as ethylenediaminetetraacetic acid, a buffer such as an acetate, citrate or phosphate and an agent for the adjustment of tonicity such as sodium chloride or dextrose, and the like, for example. In further embodiments, the pH may be adjusted with acids or bases, such as hydrochloric acid or sodium hydroxide. Parenteral preparations may be enclosed in ampoules, syringes, multiple dose vials made of glass or plastic, and the like, for example.

Pharmaceutical compositions suitable for injection include sterile aqueous solutions or dispersions and sterile powders for the extemporaneous preparation of sterile injectable solutions, dispersions, and the like, for example. For intravenous administration, suitable carriers include physiological saline, bacteriostatic water, Cremophor ELTM (BASF, Parsippany, N.J.), phosphate buffered saline (PBS), and the like, for example. In other embodiments, the carrier can be a solvent or dispersion medium containing water, an alcohol such as ethanol, a polyol such as glycerol, propylene glycol, and liquid polyethylene glycol, suitable mixtures thereof, and the like, for example. In certain embodiments, these pharmaceutical compositions are fluid to the extent that easy syringability exists. The proper fluidity may be maintained by the use of a coating such as lecithin, or by the use of surfactants, and the like, for example. In more particular embodiments, pharmaceutical compositions for injection are preserved against the contaminating action of microorganisms, such as bacteria, fungi, and the like. Prevention of the action of microorganisms may be achieved by various antibacterial and antifungal agents such as parabens, chlorobutanol, phenol, ascorbic acid, thimerosal, and the like, for example. In certain embodiments, isotonic agents such as sugars, polyalcohols such as manitol, sorbitol, sodium chloride can be used in the nitroxide containing composition. Prolonged absorption of the injectable compositions may be brought about by including an agent which delays absorption such as aluminum monostearate, gelatin, and the like, for example.

Injectable solutions, to be used with the methods herein, can be prepared by any available processes known in the art. Detail on how to prepare injectable solutions is provided in Remington's Pharmaceutical Sciences, 18^{th} ed. 1990, which is hereby incorporated by reference in its entirety. In some embodiments, injectable solutions can be prepared by incorporating nitroxide in the desired amount in an appropriate solvent alone, or with one or more additional ingredients enumerated herein, or known in the art. In further embodiments, the solution can be filtered sterilized after dissolving the nitroxide.

In other embodiments, nitroxide containing dispersions can be prepared according to any available process. Detail on how to prepare injectable dispersions is provided in Remington's Pharmaceutical Sciences, 18^{th} ed. 1990, which is hereby incorporated by reference in its entirety. In certain embodiments, injectable dispersions can be prepared by incorporating nitroxide into a sterile vehicle containing a basic dispersion medium, alone, or with one or more additional ingredients, such as those provided herein or known in the art, for example.

Other embodiments involve using nitroxides that are in a low-residue formulation. Developing low-residue formulations can be done by preparing solutions of nitroxides in low-residue gels, thickened liquids, liquids and the like. Further embodiments include nitroxides in a formulation with a sufficient viscosity such that the formulation does not immediately run off the treated area upon application. Developing formulations with sufficient viscosity can be done by preparing solutions of nitroxides in gels, liquids, thickened liquids, emulsions, dispersions, and suspensions for example.

Embodiments herein include methods of using topical formulations containing a nitroxide. In particular embodiments, topical formulations can be prepared such that they can readily be applied to all areas of a patients skin, including the scalp, face, neck, chest, arms, legs, torso, back, and the like. Topical formulations can also be prepared such that they can be applied to all mucous membranes of a patient including areas of the eyes, mouth, nose, vagina, rectum, and the like. In certain embodiments, it is preferred that formulations used to treat mucous membranes include water, or another non-irritating solvents. In additional embodiments, the formulations to be applied to mucous membranes lack irritating solvents such as alcohol, urea, and the like.

In topical formulations, the total quantity of a nitroxide or other active ingredients absorbed can vary greatly based on many factors including application area size, the frequency and vigor of application, and the viscosity or thickness of the applied vehicle. Other factors influencing drug absorption are the application site, age and condition of the skin. For example, non-keratinized, aged, broken or abraded skin will result in higher drug absorption, because these skin types are more readily penetrated by an active ingredient. Accordingly, one embodiment herein is to optimize the absorption of a nitroxide by the treated patient.

Other embodiments include topical formulations with sufficient viscosity. In certain embodiments, the pharmaceutical composition should have a viscosity that keeps the nitroxide and other active ingredients in contact with the treated area for a sufficient period of time to allow suitable absorption to the treated area. In some embodiments, formulations can have a suitable viscosity such that the formulation will not immediately run off the treated area. Accordingly, methods of retaining the formulation in place are encompassed herein.

Alternative embodiments include topical formulations with low viscosity, including, but not limited to, liquids and thickened liquids. In some embodiments, liquids and thickened liquids can be applied with the aid of an applicator to allow suitable application of the nitroxide to the treated area. Applicators can include, but are not limited to, cloths, rags, sponges, towels, gauze, and like absorbent materials, and the combination of the applicator and the nitroxide solution is one aspect of the methods described herein.

In addition to including nitroxide and a solvent, the topical compositions herein can also include polymers, colorants, anti-microbials, preservatives, antioxidants, alcohols, emollients, additional active ingredients, ingredients that enhance the permeability of the treated area, water, and other ingredients commonly used in topical formulations.

Those with skill in the art can readily modify the thickness of nitroxide formulations, with polymers. Embodiments include formulations including one or more suitable polymers with moderate to high degree of compatibility with the solvent used to dissolve the nitroxide, for example. In certain embodiments, the polymers can be selected from ethylene polymers, acrylic polymers, polyvinylpyrrolidones (PVPs), polyvinyl copolymers, cellulose polymers, including modified cellulose, natural polymers including collagen, polystyrene polymers, silicone polymers, inorganic polymers, and the like.

Examples of ethylene polymers that can be used include, but are not limited to, oxidized polyethylene, polyethylene, polyethylene glycol, and the like.

Examples of acrylic polymers that can be used include, but are not limited to, acrylic esters, methacrylic esters copolymer, acrylic polymer emulsion, carbomer, ethylene acrylates, methacryiol ethyl betaine, methacrylates copolymer, octylacrylamide, acrylates, butylaminoethyl methacrylate copolymer, polyacrylamidomethylpropane sulfonic acid, polyquaternium-5, polyquaternium-6, polyquaternium-7, polyquaternium-15, and the like.

Examples of polyvinylpyrrolidones (PVPs) include, but are not limited to, polyquaternium-11, polyvinylpyrrolidone (PVP), PVP/dimethylaminoethylmethacrylate copolymers, PVP/Elcosene copolymer, PVP/ethyl methacrylate/methacrylic acid terpolymer. PVP/hexadecene copolymer, PVP/VA copolymers, styrene/PVP copolymer, and the like.

Examples of polyvinyl copolymers include, but are not limited to, ethylene vinyl acetate copolymer, PVM/MA copolymer esters, vinyl acetate/crotonic acid copolymer, vinyl acetate/crotonic acid/methacryloxybenzophenone-1 copolymer, vinyl acetate/cotonic acid/vinyl neodecanoate copolymer, carboxymethyl cellulose, hydroxyethyl cellulose, hydroxypropyl cellulose, hydroxypropyl methylcellulose, PEG celluloses, polyquaternium-4, polyquaternium-10, and the like.

Examples of natural polymers include, but are not limited to, acacia, agar, alginate, carrageenan, furcelleran, gelatin, ghatti gum, glycosaminoglycans, guar gum, guar gum derivative, hydroxypropyl guar, hyaluronic acid, karaya, locust bean gum, maltodextrin, pectin, tragacanth gum, xanthan, and the like.

Examples of polystyrene polymers include, but are not limited to, sodium polystyrene sulfonate.

Examples of silicone polymers include, but are not limited to, amino bispropyl dimethicone, cyclomethicone, dimethicone, dimethicone copolyol, hexamethyldisiloxane, methicone, octadecyl dimethicone, phenyl dimethicone, stearoxy dimethicone, and the like.

Examples of inorganic polymers, include but are not limited to bentonite, modified bentonite, magnesium aluminum silicate, modified hectorite, sodium magnesium silicate, and the like.

The above listed polymers can be used in all topical compositions and formulations described herein, including liquids, thickened liquids, gels, emulsions, dispersions, and suspensions.

### Suitable Solvents for Nitroxides

Nitroxides, such as Tempol, are readably soluble in aqueous solutions. In some embodiments, a nitroxide can be dissolved in a solvent and prepared into a formulation including gels, thickened liquids, liquids, and the like. Those skilled in the art will readily appreciate that any water miscible liquid, at appropriate levels, can be used as a solvent, including, but not limited to, glycerin, PEG's, polysorbates, and the like.

The following is a non-exclusive list of solvents that can be used for nitroxides: water, urea, alcohols and glycols. Any alcohol capable of dissolving nitroxides can be used in the formulations and methods described herein; examples include methanol, ethanol, propanol, butanol and the like. Likewise, any glycol capable of dissolving nitroxides can be used in the formulations and methods described herein; examples include ethylene glycol, propylene glycol and the like. In one preferred embodiment, the solvent not only dissolves the nitroxide, but also facilitates transdermal delivery. Thus, transdermal-delivery-facilitating agents, particular those that disrupt or solubilize components of the stratum corneum, are particularly preferred. In other embodiments, various alcohols that facilitate penetration of nitroxides into the skin can be used with the methods herein. Additional embodiments include available transdermal enhancers that allow for systemic treatment of a patient.

In certain embodiments, of the invention, the concentration of the active ingredient, a nitroxide, can be at a concentration level at or near its solubility limit. For example a nitroxide can be about 80%, 81%, 82%, 83%, 84%, 85%, 86%, 87%, 88%, 89%, 90%, 91%, 92%, 93%, 94%, 95%, 96%, 97%, 98%, 99% and 100% of saturation in the solution. Embodiments also include formulations where a nitroxide is soluble enough in the solvent to promote its release at the desired rate upon application to the treated area. All of the above described solvents can be used with the solutions described herein, including gels, thickened liquids and liquids and the like.

### Gels

As discussed above, in some embodiments, the nitroxide containing pharmaceutical composition is a topical formulation in the form of a gel. As used herein, a gel relates to a semisolid system of either suspensions made up of small inorganic particles or large organic molecules interpenetrated by a liquid. Generally, if left undisturbed for some time, gels can be in a semisolid or gelatinous state. With some gels, small amounts of water can separate on standing.

Those with skill in the art will readily know how to prepare gels. Detail on how to prepare such gels is provided in Remington's Pharmaceutical Sciences, 18^{th} ed. 1990, which is hereby incorporated by reference in its entirety. In one embodiment a gel can be prepared by slowly dispersing one or more suitable polymers in the requisite amount of suitable solvents. A discussion of suitable solvents and polymers is provided above. According to one method of preparation a polymer and a solvent can be stirred until the polymer is completely dissolved. Water can be added to the polymer/solvent solution as it is being stirred. A sufficient amount of a nitroxide can be added to the stirred mixture until the nitroxide is adequately dissolved.

Gels can be one-phase or multiple phase systems. A gel mass consisting of a network of small discrete particles is generally termed a two-phase system while singe-phase gels typically consist of organic macromolecules distributed uniformly throughout a liquid in such a manner that no apparent boundaries exist between the dispersed macromolecules and the liquid.

In certain embodiments, the gel can be a hydroalcoholic gel. In some embodiments, an alcohol such as ethanol can be used to dissolve the nitroxide while avoiding the use of solubilizers such as PEG-40, hydrogenated castor oil, polysorbate 20 or similar ingredients. The absence of these solubilizers can greatly improve the cosmetic feel of the product as the stickiness and rubbery feel can be virtually absent. In embodiments where the pharmaceutical composition has a significant alcohol (e.g., ethanol) content, additional preservation may not be required.

Those with skill in the art can use numerous methods to readily prepare hydroalcohol gels with the formulation characteristics described herein. According to one method of preparing hydroalcohol gels, a solution can be prepared by dissolving the nitroxide in ethanol. The nitroxide/ethanol solution can be added to a hydrogel. According to certain embodiments, the nitroxide/ethanol solution can be added to a premade hydrogel using a slow moving anchor mixer, which can reduce the creation of air bubbles in the hydroalcohol gel.

Due to reduced hydrogen bonding, the viscosity of a hydroalcoholic gel is generally lower than the viscosity of a corresponding hydrogel. Regardless those with skill in the art can adjust the ingredients of the hydroalcoholic gel to prepare a composition with a suitable viscosity for the desired result. For example the use of the thickening agents or polymers discussed above can be used to raise the viscosity of a particular formulation.

In some embodiments, the gel can be sprayable. Methods of preparing sprayable gels are well known in the art. According to one embodiment of preparing a sprayable gel, a suitable polymer can be added to water. Upon hydration and development of structure, the thickened polymer/water mixture can be added to a nitroxide/solvent solution.

### Liquid Formulations

Further embodiments herein include nitroxide-containing liquid formulations to prevent or ameliorate the effects resulting from a photosensitizer or sonosensitizer. For example, a nitroxide can be dissolved in any of the suitable solvents discussed herein. The following is a non-exclusive list of solvents that can be used as a solvent for Tempol: water, urea, alcohols, glycols and the like. These liquid formulations can be used with the aid of an applicator such as a towel, cloth, rag, sponge, gauze or like absorbent material in order to apply the formulation to a patient in need.

Further embodiments include adding polymers to thicken nitroxide containing liquid solutions. Any of the above described polymers can be used as a thickener for these formulations. For example, the following polymers can be used as thickening agents ethylene polymers, acrylic polymers, polyvinylpyrrolidones (PVPs), polyvinyl copolymers, cellulose polymers, natural polymers, polystyrene polymers, silicone polymers, inorganic polymers, and the like.

Those with skill in the art will readily know how to prepare thickened liquid solutions according to the methods described herein. Detail on how to prepare such liquids is provided in Remington's Pharmaceutical Sciences, 18^{th} ed. 1990, which is hereby incorporated by reference in its entirety. When the formulations described herein is practiced with a thickened liquid, it is advantageous to thicken the liquid to a viscosity of 20-100,000 or more centipoise.

### Methods of Using Compositions

Nitroxides can be administered to a patient according to any available method, including orally, topically, or parenterally, including injection, and the like, for example. Oral administration can be in the form of tablets, syrup, gel capsules, solutions, and the like, for example. Injection can be subcutaneous, intravenous, or by intramuscular injection, and the like, for example.

Suitable areas for topically applying the nitroxide formulations described herein include all areas of the skin and mucous membranes. Methods include, but are not limited to, applying formulations to the scalp, face, neck, chest, arms, legs, torso, back, and the like. Further methods include, but are not limited to, applying the formulations to mucous membranes, including but not limited to, areas of the mouth, nose, eyes, vagina, rectum and the like.

Some embodiments include rubbing a nitroxide-containing formulation onto an area of a susceptible patient, in order to facilitate the absorption of the nitroxide. Rubbing can be accomplished using the practitioner's hands, typically gloved, or alternatively be done with an applicator such as a cloth, towel, sponge, rag, gauze and the like. Alternatively, upon being applied on the treated area, the formulation may be left alone to absorb. Specific embodiments include topically applying a sufficient amount of a nitroxide such as 4-hydroxy-2,2,6,6-tetramethylpiperidine-1-oxyl to prevent or ameliorate the negative effects of photosensitizers and sonosensitizers.

Any dose of a particular nitroxide that is capable of preventing or ameliorating the effects of a photosensitizer or sonosensitizer can be used with the methods described herein. In certain embodiments, the nitroxide can be used at a dose of about 1, 1.5, 2, 2.5, 3, 3.25, 3.5, 3.75, 4, 4.25, 4.5, 4.75, 5, 5.25, 5.5, 5.75, 6, 6.25, 6.5, 6.75, 7, 7.25 7.5, 7.75, 8, 8.25, 8.5, 8.75, 9, 9.1, 9.2, 9.3, 9.4, 9.5, 9.6, 9.7, 9.8, 9.9 and 10 mg/kg, for example. In other embodiments, the dose of the nitroxide can be about, 10.5, 11, 11.5, 12, 12.5, 13, 13.5, 14, 14.5, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, and 300 mg/kg, for example.

In some embodiments, a nitroxide can be administered to a patient immediately after phototherapy, photodiagnosis, or SDT, or some time afterwards. In certain embodiments, the nitroxide can be administered about 8, 7, 6, 5, 4, 3, or 2 weeks after the patient undergoes phototherapy, photodiagnostics, or SDT. In other embodiments the nitroxide can be administered about 13, 12, 11, 10, 9, 8, 7, 6, 5, 4, 3, or 2 days after the patient undergoes phototherapy, photodiagnostics, or SDT. In still further embodiments, the nitroxide can be administered about 24, 23, 22, 21, 20, 19, 18, 17, 16, 15, 14, 13, 12, 11, 10, 9, 8, 7, 6, 5, 4, 3, or 2 hours after the patient undergoes phototherapy, photodiagnosis, or SDT. In other embodiments, a nitroxide can be applied to a patient about 119, 118, 117, 116, 115, 110, 105, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, or 2 minutes after the patient undergoes phototherapy, photodiagnosis, or SDT. In other embodiments, a nitroxide can be applied to a patient about 119, 118, 117, 116, 115, 110, 105, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, or 2 seconds after the patient undergoes phototherapy, photodiagnosis, or SDT. In particular embodiments, the nitroxide can be administered to a subject prior to the subject being exposed to light, in order to prevent burning.

In some embodiments, the nitroxide can be administered in 1, 2, 3, 4, 5, 6, 7, 8, 9 or 10 doses. In other embodiments, the nitroxide can be administered about 1, 2, 3, 4, 5, 6, 7, 8, 9, or about 10 times daily. Specific embodiments include regular (e.g., monthly, twice monthly, weekly, twice weekly, thrice weekly, daily, twice daily, thrice daily) administration to a patient who has undergone phototherapy, photodiagnostics, SDT, and the like, for example. In other embodiments, the nitroxide can be administered after about one or two times the half life of the nitroxide, for example.

Method embodiments include the use of any nitroxide to ameliorate the negative effects caused by a photosensitizer or sonosensitizer in a patient's body. As used herein the term “ameliorate” includes methods of prevention or treatment. The terms “effects,” “harmful effects,” and “negative effects” are to be construed broadly and relate to any harmful result (both indirect and direct) from the use of photosensitizers or sonosensitizers and include, for example, oxidative stress, necrosis, photosensitivity (which can lead to burning), general toxicity, phototoxicity, apoptosis, and subcellular damage including damage to organelles, DNA, RNA and the like, for example. In certain embodiments, the methods herein can be used in conjunction with a currently available method of phototherapy (e.g., PDT) and SDT. In other embodiments, the methods herein can be used in conjunction with treatment methods that will be developed in the future. In other embodiments, the methods provided herein can be used in conjunction with photodiagnosis, both methods that are currently available and those that will be available in the future.

As phototherapy (e.g., PDT), SDT, and photodiagnosis can be applied to a wide variety of different diseases and conditions, the methods described herein include the use of a nitroxide to ameliorate the negative effects of a photosensitizer or sonosensitizer used to treat, prevent or diagnose the following non-exclusive list of diseases and conditions in a patient: adenoma of the prostate gland, transplant rejections (e.g., using sensitizers to kill immune cells), benign prostatic hypertrophy, chronic prostatitis, otorhinolaryngologic diseases, (e.g., sinusitis, frontitis, polyposis), neovascular ophthalmic diseases (e.g., wet AMD, diabetic retinopathy, neovascular retinal diseases, central retinal vein occlusion, rubeosis iridis, herpes simplex, keratitis, trachoma, ptenygium histoplasmosis, subfoveal choroidal neovascularization), atherosclerotic plaques (e.g., photoangioplasty), peridontitis, chronic and acute gingivitis, alveolitis, autoimmune diseases (e.g., using sensitizers to kill immune cells that can cause multiple sclerosis, rheumatoid arthritis), septicemia, bacterial infections, yeast infections, viral and inflammatory diseases, cervicitis, endometriosis, uterine fibroids, genital verucca, warts, pelvic inflammatory disease, Chlamydia disease, pre-malignant, carcinoma in situ of the cervix, acne, rosacea, psoriasis, herpes, papillomas, suppurative wounds, ulcers, herpes zoster, seborrheac dermatitis, leucoplakia, histoplasmosis, coccidiomycosis, hair removal, mole removal, keloid scars, tattoos, joints diseases (e.g., rheumatoid arthritis, osteomyelitis), hormone deficiency, mental depression, veterinary diseases (e.g., cancer, suppurative wounds, ulcers), viral infections (e.g., human immunodeficiency virus type I, herpes simplex virus type I/II, human cytomegalovirus, measles, simian virus, papilloma virus) and leukemia.

In other embodiments, the methods herein include the use of a nitroxide to ameliorate the negative effects of a photosensitizer or sonosensitizer used to treat, prevent or diagnose tuberculosis, leprosy, malaria, oncocerciasis, and other like tropical diseases.

In certain embodiments, the methods herein include the use of a nitroxide to ameliorate the negative effects of a photosensitizer or sonosensitizer used to treat, prevent and diagnose any type of cancer (including tumors and precancerous conditions) including, but not limited to, cancer of the brain, larynx, lung, oral cavity, breast, ovaries, testicles, skin (e.g., melanoma, basal and squamous cell carcinoma), esophagus, stomach, gall bladder, cervix, bone, bladder, blood (e.g., leukemia), head and neck.

Further embodiments include topically applying a sufficient amount of a nitroxide such as 4-hydroxy-2,2,6,6-tetramethylpiperidine-1-oxyl to prevent or treat a harmful side effect caused by a photosensitizer or sonosensitizer.

### EQUIVALENTS

The foregoing description details certain preferred embodiments of the teachings herein and describes the best mode contemplated by the inventors. It will be appreciated, however, that no matter how detailed the foregoing may appear in text, the methods of using a nitroxide to ameliorate the effects of a photosensitzer or sonosensitizer can be practiced in many ways and the teachings herein should be construed in accordance with the appended claims and any equivalents thereof. The foregoing written specification is considered to be sufficient to enable one skilled in the art to practice the embodiments described herein.

What is claimed is:

1. A method of treating a patient, comprising
administering a photosensitizer or sonosensitizer to the patient; and
administering a sufficient amount of a nitroxide to ameliorate a negative side effect resulting from said photosensitizer or sonosensitizer.

2. The method of claim 1, further comprising administering photodynamic therapy to the patient.

3. The method of claim 1 wherein the photosensitizer is selected from the group consisting of porphyrins, chlorins, and phthalocyanines.

4. The method of claim 1, wherein the photosensitizer is porfimer sodium.

5. The method of claim 1, wherein the nitroxide is 4-hydroxy-2,2,6,6-tetramethylpiperidine-1-oxyl (TEMPOL).

6. The method of claim 1, wherein the negative side effect is oxidative stress.

7. The method of claim 1, wherein the negative side effect is photosensitivity.

8. The method of claim 1, wherein the negative side effect is generalized toxicity.

9. The method of claim 1, wherein the negative side effect is cellular apoptosis.

10. The method of claim 1, further comprising
applying ultrasound or sonoluminescence to a target region on the patient.
